(19) United States
(12) Patent Application Publication
(10) Pub. No.: US 2010/0035801 A1
(43) Pub. Date: Feb. 11, 2010

(54) METHOD FOR ACCELERATING CUTANEOUS BARRIER RECOVERY

(75) Inventors: Mitsuhiro Denda, Yokohama-shi (JP); Kaori Inoue, Yokohama-shi (JP)

Correspondence Address: FISH & RICHARDSON PC, P.O. BOX 1022, MINNEAPOLIS, MN, 55440-1022, US

(73) Assignee: Shiseido Company Ltd., a Tokyo Japan corporation

(21) Appl. No.: 12/581,823

(22) Filed: Oct. 19, 2009

Related U.S. Application Data
(63) Continuation of application No. 11/797,997, filed on May 9, 2007.
(60) Provisional application No. 60/799,659, filed on May 12, 2006.

(51) Int. Cl. A61K 38/12; A61K 31/505; A61K 31/54; A61P 17/00

(52) U.S. Cl. 514/11; 514/275; 514/222.5

(57) ABSTRACT

A method for accelerating cutaneous barrier recovery by inducing efflux of potassium ion from an epidermal cell as well as a method for preventing epidermal hyperplasia induced by inducing efflux of potassium ion from an epidermal cell are provided.

### FIELD OF THE INVENTION

The present invention relates to a method for accelerating cutaneous barrier recovery.

### BACKGROUND ART

A critical function of the skin of terrestrial mammals is the generation of a water-impermeable barrier against excess transcutaneous water loss. The importance of the barrier for the organism is shown by a series of homeostatic processes that immediately accelerate after the epidermal permeability barrier is damaged, thereby rapidly restoring barrier homeostasis (Elias, P. M. and K. R. Feingold (2001). Arch Dermatol 137(8): 1079-81). These processes include increased lipid synthesis, augmented lipid processing, and the acceleration of the exocytosis of lamellar body contents, including the extracellular lipids that form the barrier. Moreover, permeability barrier status is linked to the pathophysiology of inflammatory dermatoses, because defective barrier function parallels the severity of clinical phenotype in clinical dermatology (Elias, P. M., Wood L. C., Feingold K. R. (1999). Am J Contact Dermat 10(3): 119-26.).

Our laboratories have focused on the role of changes in extracellular ions as potential regulatory signals of permeability barrier homeostasis. We previously demonstrated that influx of calcium ions into epidermal keratinocytes inhibits lamellar body (LB) secretion and delays epidermal permeability recovery after barrier disruption (Lee, S. H., Elias P. M., Feingold K. R. et al. (1992). J Clin Invest 89(2): 530-8; Denda, M., Fuziwara S, Inoue K. (2003). J Invest Dermatol 121(2): 362-7; Mauro, T., Bench, Sidderas-Haddad E., Elias P. M., Feingold K R. Cullender C (1998). J Invest Dermatol 111(6): 1198-201.) On the other hand, influx of chloride ions into epidermal keratinocytes accelerates LB secretion and consequently barrier recovery rate after barrier disruption. These results suggest that electro-physiological balance between the outside and inside of keratinocytes cell membranes is an important influence on barrier homeostasis. We recently demonstrated several similarities between neurons and keratinocytes (Denda, M., Inoue K., Inomata S., Denda S. (2002). J Invest Dermatol 119(5): 1041-7; Fuziwara, S., Inoue K., Denda M. (2003). J Invest Dermatol 120(6): 1023-9), perhaps because both cell types derive from the ectoderm during the early stages of embryonic development. Potassium influx/efflux is also an important signaling system, which regulates stress responses in neurons (Shepherd, G. M. (1994). Neurobiology. Oxford, UK, Oxford University Press: 132-159). Analogously, in epidermal keratinocytes, increased extracellular K⁺ also inhibits LB exocytosis, synergistically and in parallel with changes induced by calcium Lee, S. H. et. al., op. cit.; Mauro, T, et. al., op. cit.), presumably due to increased intracellular K⁺ levels.

### DISCLOSURE OF INVENTION

Thus, we hypothesized that skin barrier homeostasis could be modulated by changes in intracellular potassium levels, occurring via K⁺ channels.

We evaluated effects of regulators of K⁺ channels on hairless mice barrier homeostasis, and found that single applications of either K⁺ channel openers (i.e., 1-EB10, minoxidil, diazoxide) or the K⁺ ionophore, valinomycin, accelerate barrier recovery after acute insults to murine skin, paralleled by a reduction in intracellular K⁺ levels in CHK. In contrast, applications of K⁺ channel blockers (i.e., gilbenclamide, dequalinium) delay barrier recovery. Alterations in intracellular K⁺ regulate barrier homeostasis by either stimulating (reduced K⁺) or inhibiting (elevated K⁺) lamellar body secretion. Finally, development of epidermal hyperplasia, a down-stream consequence of barrier disruption, is also inhibited by agents that reduce intracellular K⁺ levels.

These results demonstrate that changes in K⁺ levels that can be presumed to occur after barrier disruption, signal metabolic responses; i.e., lamellar body secretion, which accelerates normalization of barrier function.

Accordingly, in the first aspect, the present invention provides a method for accelerating cutaneous barrier recovery by inducing efflux of potassium ion from an epidermal cell.

In the second aspect, the present invention provides a method for preventing epidermal hyperplasia induced by inducing efflux of potassium ion from an epidermal cell.

### BRIEF DESCRIPTIONS OF DRAWINGS

FIG. 1 shows the effects of topical potassium channel openers and blockers on permeability barrier recovery after tape stripping. Topical application of potassium channel openers, 1-EB10, minoxidil, and diazoxide to hairless mice flanks after stripping significantly accelerated barrier repair. On the other hand, application of potassium channel blockers, gilbenclamide and dequalinium significantly delayed barrier recovery. Concentration of each reagent was 1 mM. These effects were observed 1 (A), 3 (B) and 6 hours (C) after the treatment. Four animals were used for each treatment, and eight for controls. Two points were measured on each flank. The results of ANOVA test show F value is 99.972 and probability is less than 0.0001. The results of statistical differences are as follows. *:P<0.05, **:P<0.005, ***:P<0.0005.

FIG. 2 shows the effects of topical potassium ionophore, valinomycin, on permeability barrier recovery. Either tape stripping (A) or acetone treatment (B) was performed on hairless mouse flanks. Valinomycin accelerated barrier recovery after both tape stripping and acetone treatment (n=4 for each treatment; and 2 points were measured on each flank.) The results of ANOVA test are shown at the bottom of each figure. The results of statistical differences are as follows. *:P<0.05, ***:P<0.0005.

FIG. 3 shows the changes in intracellular potassium (K⁺) concentration induced in cultured human keratinocytes by a K⁺ channel opener and ionophore. Treatment with two different types of potassium channel openers, Ca⁺⁺-dependent type, 1-EB10 (10 μM), and ATP-dependent type, diazoxide (10 μM) decreased intracellular K⁺ concentrations. Treatment with valinomycin (1 μM) also decreased intracellular K⁺ concentrations. For each reagent, experiments were performed 3 times with 10 cells chosen from each experiment for measurements and quantification. The results of ANOVA test are shown on the bottom of each graph.

FIG. 4 shows that accelerated lamellar body secretion accounts for rapid barrier recovery with K⁺ openers and ionophore. A: Untreated, control (Co) mouse epidermis prior to acetone treatment. Note substantial secreted lamellar body (LB) contents at and just below the stratum granulosum (SG)-stratum (SC) junction. B: One hour after acetone (A) treatment and vehicle applications much of the secreted lamellar material has been extracted (asterisks) and not yet restored from the pool of intracellular LB (arrowheads). C & D: One hour after acetone treatment plus diazoxide (D) or valinomycin (V) treatment much more secreted material is evident at SG-SC interface (arrows), extending to deeper SG layers (partially shown). Finally, in contrast to vehicle-treated skin, the cytosol of outmost SG cells is devoid of LB. Skin sections were stained with osmium tetroxide. Bars in each photograph are 0.5 μm.

FIG. 5 shows that topical application of potassium channel opener, Diazoxide prevented the epidermal hyperplasia induced by acetone treatment under low environmental. The representative sections are shown in FIGS. 5A and 5B. FIG. 5A shows the hyperproliferative epidermis treated with water after the acetone treatment under the low humidity. Dark spots show BrdU positive cells. The increase was seen on the epidermal basal layer (FIG. 5A). Topical application of 100 uM Diazoxide prevented the epidermal hyperplasia (FIG. 5B).

FIG. 6 illustrates a quantified results shown in FIG. 5. We used 6 mice for control and 5 for Diazoxide treatment. The results of ANOVA test show F value is 5.968 and probability is 0.0372.

### BEST MODE FOR CARRYING OUT THE INVENTION

To test that skin barrier homeostasis could be modulated by changes in intracellular potassium levels, occurring via K⁺ channels, we employed a number of chemically different drugs that alter intracellular K⁺ levels. If a variety of different drugs that increase intracellular K⁺ levels have similar effects on barrier homeostasis that would strongly suggest that the effect is due to changes in K⁺ and not due to other effects of the various drugs. Moreover, if drugs that decrease intracellular K⁺ have opposite effects on barrier homeostasis compared to drugs that increase K⁺ that would provide further support that the changes are specifically due to changes in intracellular K⁺ and not other non specific effects.

The drugs used in this study include:

1) Diazoxide, an anti-hypertensive ATP-dependent K⁺ channel opener (7-chloro-3-methyl-2H-1,2,4-benzothiadiazine 1,1-dioxide: C₂H₇ClN₂O₂S), that inhibits insulin secretion in pancreatic beta-cells (Trube G., Rorsman P., Ohno-Shosaku T. Arch 407:493-499, 1886);

2) Minoxidil, another ATP-dependent K+ channel opener (6-(1-Pipendinyl)-2,4-pyrimidinediamine 3-oxide: C₉H₁₅N₅O) originally reported to cause vasodilatation (Meisheri K D., Khan S A., Martin J L. J Vasc Res 30: 2-12, 1993);

3) 1-EB10 (1-Ethyl-2-benzoimidazollnone: C₉H₁₀N₂O), a different type of K⁺ channel opener, which was the first epithelial Ca²⁺ dependent K⁺ opener to be discovered (Devor D C, Singh A K, Frizzel R A., Am J Physiol 271:L775-784, 1996);

4) Gilbenclamide (5-chloro-N {2-[4-[[[cyclohexylamino]carbonyl]amino]sulfonyl]phenyl}-2-metyhoxybenzamide: C₂₃H₂₈ClN₃O₃S), an ATP-dependent K⁺ channel blocker originally reported to increase intracellular Ca²⁺ and stimulate insulin secretion (Robertson D. W., Schober D. A., Krushinski J. H., Mais D. E., Thompson D. C., Gehlert D. R., J Med Chem 33:3124-3126, 1990);

5) Dequalinium (1,1-(1,10-Decanedyl) bis(4-amino-2-methylquinolinium) dichloride: C₃₀H₄₀Cl₂N₄), another type of K+ channel blockers, which is Ca²⁺-activated and a selective blocker of the hyperpolation in rat neurons (Dunn P M. Eur J Pharmacol 252:189-194, 1994); and

6) Valinomycin, a potassium ionophore (Inai Y., Yabuki M., Kanno T., Akiyama J., Yasuda T., Utsumi K. (1997) Cell Struct Funct 22:555-563).

As a result, we demonstrated that changes in K⁺ levels that can be presumed to occur after barrier disruption, signal metabolic responses; i.e., lamellar body secretion, which accelerates normalization of barrier function.

The method for accelerating cutaneous barrier recovery comprises the step of inducing the efflux of potassium ion from cell, inter alia, epidermal cell, for example, by applying a potassium channel opener and/or a potassium ionophore on skin.

The method for preventing epidermal hyperplasia induced by barrier disruption comprises the step of inducing the efflux of potassium ion from cell, inter alia, epidermal cell, for example, by applying a potassium channel opener and/or a potassium ionophore, on skin.

As the potassium channel opener, a number of compounds are known in the art, and examples thereof include, but not limited to, 1-EB10, minoxidil, diazoxide and the like.

As the potassium ionophore, a number of compounds are known in the art, and examples thereof include, but not limited to, valinomycin, nigericin and the like.

The above compounds themselves can be active ingredients for accelerating cutaneous barrier recovery or preventing epidermal hyperplasia induced by barrier disruption. The present invention not only ultimately leads to improvement of skin barrier functions, but can contribute to prophylaxis or treatment of dermatological diseases and cosmetic skin care.

The above compounds are used, as an active ingredient for a pharmaceutical or cosmetic composition for accelerating cutaneous barrier recovery and/or preventing epidermal hyperplasia induced by barrier disruption according to the invention, generally, as dry weight, in an amount of 0.00001 to 10% by weight, preferably 0.0001 to 5% by weight per weight of the total composition. At lower than 0.00001% by weight, the effects of the invention are hard to exert sufficiently, and even if it is compounded in an amount more than 10% by weight, so much enhancement of the effects is not attained and formulation becomes undesirably harder.

The pharmaceutical or cosmetic composition to be thus prescribed can be prepared by mixing or homogenizing the at least one of the above compounds into a suitable solvent, e.g., pure water, deionized water or buffered water, a lower alkanol such as methanol, ethanol or isopropyl alcohol or an aqueous solution thereof, glycerol or an aqueous solution thereof, a glycol such as propylene glycol or 1,3-butylene glycol or an aqueous solution thereof, or an oil such as hardened castor oil, vaseline or squalane, if necessary with use of a surfactant or the like. Into the composition can further appropriately be compounded, in such a range that the effects of the invention, that is, acceleration of cutaneous barrier recovery and/or prevention of epidermal hyperplasia induced by barrier disruption is/are not spoiled, other components usually used for external preparations such as cosmetics or pharmaceuticals, for example whitening agents, humectants, antioxidants, oily substances, ultraviolet absorbers, surfactants, thickeners, higher alcohols, powdery substances, colbrants, aqueous substances, water, various skin nutrients, etc., according to necessity. Further, into the composition of the invention can appropriately be compounded sequestering agents such as disodium edetate, trisodium edetate, sodium citrate, sodium polyphosphate, sodium metaphosphate and gluconic acid, drugs such as caffeine, tannin, verapamil, tranexamic acid and its derivatives, grabridin, extract of fruit of Chinese quince with hot water, various crude drugs, tocopherol acetate, and glycylrrhetinic acid and its derivatives or salts, whiteners such as vitamin C, magnesium ascorbate phosphate, ascorbic acid glucoside, arbutin and kojic acid, saccharides such as glucose, fructose, mannose, sucrose and trehalose, vitamin A derivatives such as retinoic acid, retinol, retinol acetate and retinol palmitate, etc.

As to the above composition, its dosage form is not particularly limited, and can be any dosage forms such as solutions, solubilizing forms, emulsified forms, dispersed powders, water-oil two layer forms, water-oil-powder three layer forms, ointments, gels or aerosols. Its use form can also be optional, and can, for example be facial cosmetics such as skin lotion, liquid cream, cream and pack, foundation, and further makeup cosmetics, cosmetics for hair, aromatic cosmetics, bathing agents, etc., but is not limited thereto.

When the above composition is used on a living body, it can be endermically administered to local skin or the whole body skin of a subject. Its dose cannot be limited because the optimal amount varies depending on the age, sex and skin state of subjects, but, usually, it is sufficient that a composition prepared as mentioned above is administered onto the skin once or several times a day. If necessary, the dose or administration frequency can be determined referring to results obtained by evaluating a suitable specimen according to the evaluation method described later.

### Examples

### Materials

All experiments were performed on 7-10-week old male hairless mice (HR-1, Hoshino, Japan). All procedures for measuring of skin barrier function, disrupting the barrier and applying the sample were carried out under anesthesia. All experiments were approved by the Animal Research Committee of the Shiseido Research Center in accordance with the National Research Council Guide. 1-EB10, diazoxide, gilbenclamide, minoxidil, dequalinium were purchased from Tocris (TOCRIS, Bristol, UK). Valinomycin was purchased from Wako (Wako Osaka, Japan).

Cutaneous Barrier Function

Permeability barrier function was evaluated by measurement of transepidermal water loss (TEWL) with an electric water analyzer, as described previously (Denda, M., Sato J., Masuda Y., Kuramoto M., Elias P. M., Feingold K. R. (1998). J Invest Dermatol 111(5): 858-63). For barrier recovery experiments, both sides of flank skin were treated with repeated tape stripping until the TEWL reached 7-10 mg per cm² per h. Immediately after barrier disruption 100 μl of an aqueous solution containing 1 μM of reagent or water alone (control) was applied to the treated area. We did not apply the same reagent to both flanks. The areas were covered with plastic membranes for 15 min (both treated and control sites) and then the membranes were removed. Previous studies have shown that occlusion for a longer period of time delays barrier repair. However, 15 min of occlusion did not affect barrier repair (data not shown). Two points on one side of a flank were measured and 4-8 mice were used to evaluate the effects of each treatment. We always disrupted the barrier between 7:00 AM to 8:00 AM to avoid variations in repair rate due to the influence of circadian rhythm. TEWL was then measured over the same sites at 1, 3 and 6 hours after barrier disruption. The barrier recovery results are expressed as percent of recovery, because of variations from day to day in the extent of barrier-disruption. In each animal, the percentage of recovery was calculated by the following formula: (TEWL immediately after barrier disruption−TEWL at indicated time point)/(TEWL immediately after barrier disruption−baseline TEWL)×100%). All experiments were performed on 7 to 10-week-old male hairless mice (HR-1, Hoshino, Japan). All procedures including measurement of skin barrier function, disruption of the barrier, and application of test samples were carried out under anesthesia.

Evaluation of Intracellular Potassium Levels

All in vitro cell culture measurements were carried out using second passage human neonatal keratinocytes. We incubated cells in a low calcium medium (0.1 mM calcium, HuMedia-KG2, KURABO, Osaka, Japan) for at least 5 days and used the cells within 10 days. These keratinocytes were incubated until 100% of confluency, incubated 3 more days, and then incubated in a high calcium medium (1.8 mM calcium) for 24 hours to induce differentiation of the keratinocytes. We then added PBF1-AM (Molecular Probes, Eugene, Oreg.), final concentration 10 μM, and then incubated the cells for 4 hours at 37 C. The cultured keratinocytes were then washed in the medium described above. The cover slip was mounted on a fluorescence microscope (IX70, TS Olympus, Tokyo, Japan) equipped with a 75 W xenon-lamp and band-path filters of 340 nm. Measurements were carried out at the room temperature. Imaging data, recorded by a high-sensitive silicon intensifier target camera (C4742, Hamamatsu Photonics, Hamamatsu, Japan) were recorded by a fluorescence analyzing system (AQUACOSMOS/RATIO1, Hamamatsu Photonics, Hamamatsu, Japan).

Electron-Microscopic Study

The full thickness of skin samples for electron microscopy was minced into pieces (<0.5 mm³) and fixed in modified Karnovsky's fixative overnight. They were then post-fixed in 2% aqueous osmium tetroxide or 0.2% ruthenium tetroxide as described previously (Denda et al. 1998, op. cit.). After fixation, all samples were dehydrated in graded ethanol solutions, and embedded in an Epon-epoxy mixture. Thin sections were stained with lead citrate and uranyl acetate and viewed by electron microscopy.

Epidermal hyperplasia induced by barrier disruption under low humidity.

Animals were kept separately in 7.2 liter cages in which the relative humidity was maintained at less than 10% with dry air as described previously (Denda et al. 1998a). The temperature was the same in all cases (22-25° C.), and fresh air was circulated 100 times per hour. Animals were kept out of the direct stream of air. During the experiments, the animal's behavior was not restricted. The level of NH₃ was always below 1 ppm. Animals were first kept in a dry condition for 48 hours and then the skin on both flank sides was treated with acetone-soaked cotton balls, as described previously (Denda et al. 1998a). The procedure was terminated when TEWL reached 2.5-3.5 mg per cm² per h. Immediately after the barrier disruption, 100 ul of Diazoxide aqueous solution (100 uM) was applied on one side of the treated area. Water was applied on the other side. Then the animals were again kept in the dry condition for 48 hours. After the experiments, animals were euthanized with diethylether inhalation and skin samples were taken from the treated area. One hour before the euthanization, 20 ul per g body weight bromodeoxyuridine (BrdU) 10 mM solution was injected intraperitoneally. Untreated control mice were also treated with BrdU at the same time. After fixation with 4% paraformaldehyde, full thickness skin samples were embedded in paraffin, sectioned (4 um), and processed for hematoxylin and eosin staining. On each section, five areas were selected at random; the thickness of the epidermis was measured with an optical micrometer, and the mean value was calculated. For the assessment of DNA synthesis, the sections were immunostained with anti-BrdU antibodies. On each section, five areas were selected at random from one section; the number of immunostained cells per 1 mm of epidermis was counted and the mean value was calculated. Measurements were carried out in an observer-blinded fashion.

Statistics

The results are expressed as the mean±SD. Statistical differences between two groups were determined by a two-tailed Student's t-test. In the case of more than 2 groups, differences were determined by ANOVA test (Fisher's protected least significant difference).

Results

Pharmacologic Agents that Modulate Intracellular Potassium Alter Permeability Barrier Homeostasis:

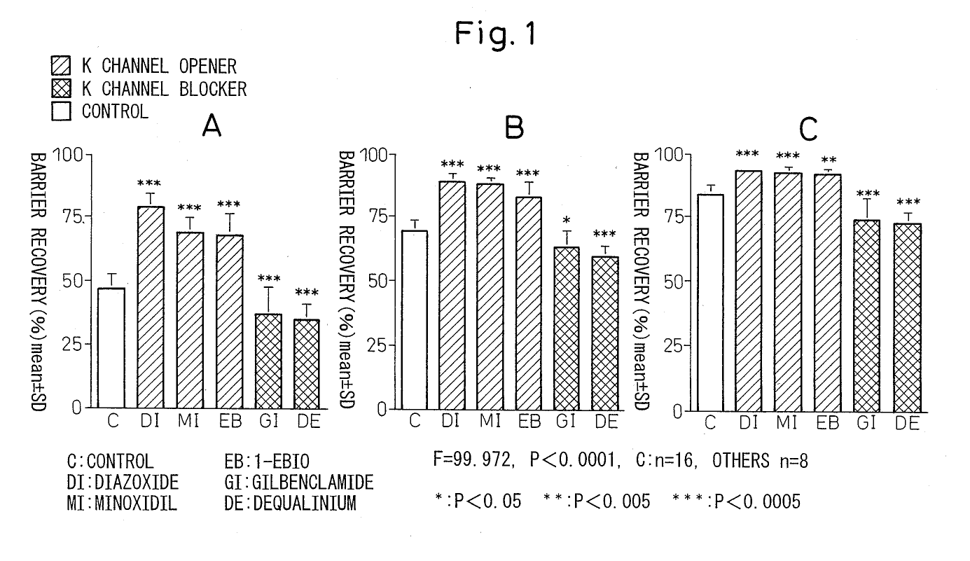

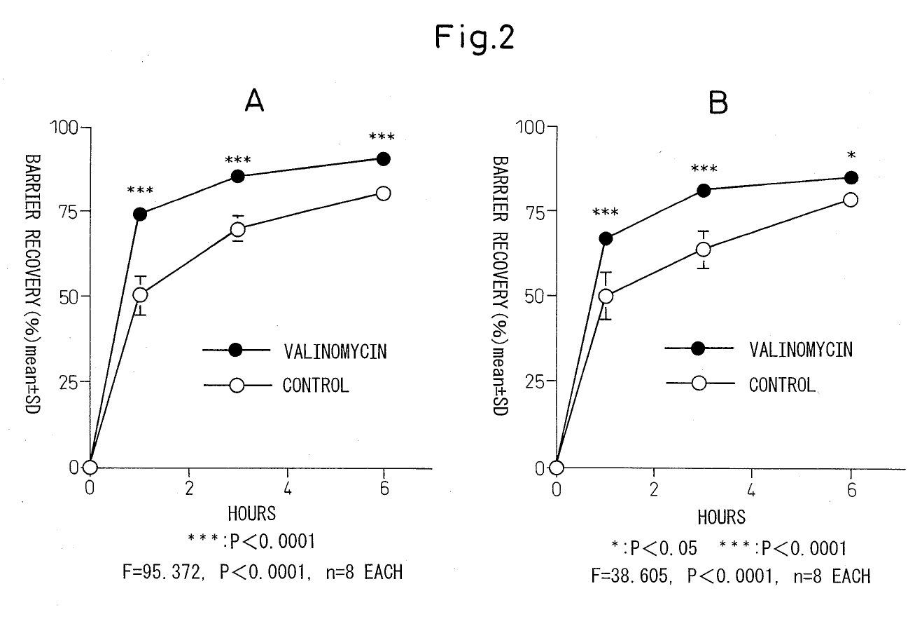

We first assessed the effects of a single topical application immediately after acute barrier disruption of three, chemically-unrelated, potassium channel openers, diazoxide (ATP-dependent), minoxidil (ATP-dependent), 1-EB10 (Ca²⁺-dependent). As seen in FIG. 1, all three agents accelerated barrier recovery. In contrast, a single application of two chemically unrelated, potassium channel blockers, gilbenclamide (ATP-dependent) and dequalinium (Ca²⁺-dependent) delayed barrier recovery (FIG. 1). These differences in barrier recovery were seen at one, three and six hours after acute disruption (FIG. 1A-C, respectively). Finally, we assessed another, unrelated method that also modulates intracellular potassium levels; i.e., topical applications of the potassium ionophore, valinomycin. As seen in FIG. 2, valinomycin treatment, like K⁺ channel openers, also accelerated barrier recovery after either tape stripping or acetone disruption of the barrier. Together, these results suggest that reductions in intracellular K⁺ accelerate barrier recovery while maintenance of intracellular K⁺ concentration delays barrier recovery.

Effects of Pharmacologic Agents can be Attributed to Altered Intracellular Potassium:

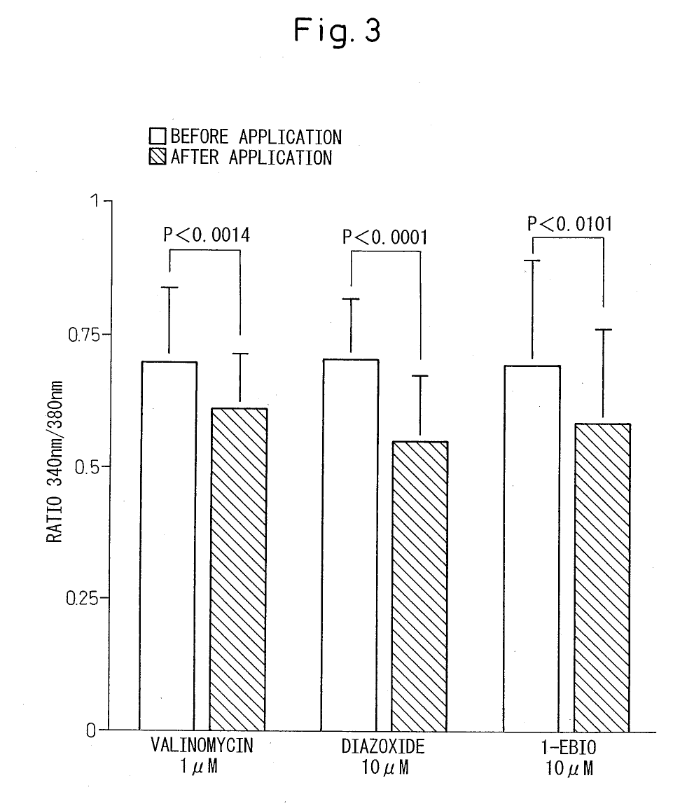

To assess whether the changes in barrier recovery reflect altered intracellular K⁺ levels, we next evaluated changes in potassium ion levels in differentiated cultured human keratinocytes (CHK) before and after application of ATP-dependent K channel opener, diazoxide (final concentration 10 mM), Ca²⁺-dependent type K channel opener, 1-EB10 (final concentration 10 μM), and K⁺ ionophore, valinomycin (final concentration 1 μM). Treatment with any of these reagents decreased intracellular potassium concentrations (FIG. 3). These results suggest that accelerated barrier recovery in response to these agents may be attributed to reduced intracellular K⁺ levels.

Improved Barrier Homeostasis Produced by Potassium-Lowering Agents Reflects Acceleration of Lamellar Body Secretion:

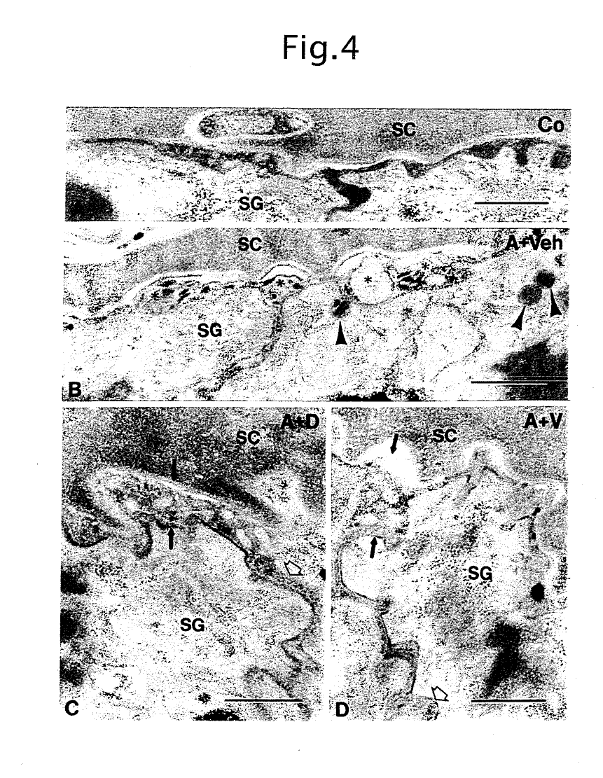

We next assessed the basis for the K⁺-induced changes in permeability barrier homeostasis. Treatment with either K⁺ channel openers, diazoxide (FIG. 4C) or the K⁺ ionophore, valinomycin (FIG. 4D), accelerated the secretion of LB from the outer layers of the stratum granulosum (SG). Accordingly, the cytosol of these SG cells was largely devoid of LB, while, in contrast, the extracellular spaces at the SG-SC interface displayed an increase in secreted LB contents (FIGS. 4C&D). In addition, these agents stimulate premature LB secretion into extracellular domains between cells of the mid SG (FIGS. 4C&D). Furthermore, accelerated LB secretion appeared to lead to a net increase in the number of mature lamellar membranes in the SC interstices (FIGS. 4C&D). In contrast to the K⁺ channel openers and K⁺ ionophore, K⁺ channel blockers inhibited LB secretion, resulting in increased density of these organelles in the cytosol of the outermost SG layers and diminished secretion of LB contents at the SG-SC interface (data not shown). Together, these results show that differences in permeability barrier homeostasis induced by agents that alter intracellular K⁺ can be ascribed to modulations in LB secretion.

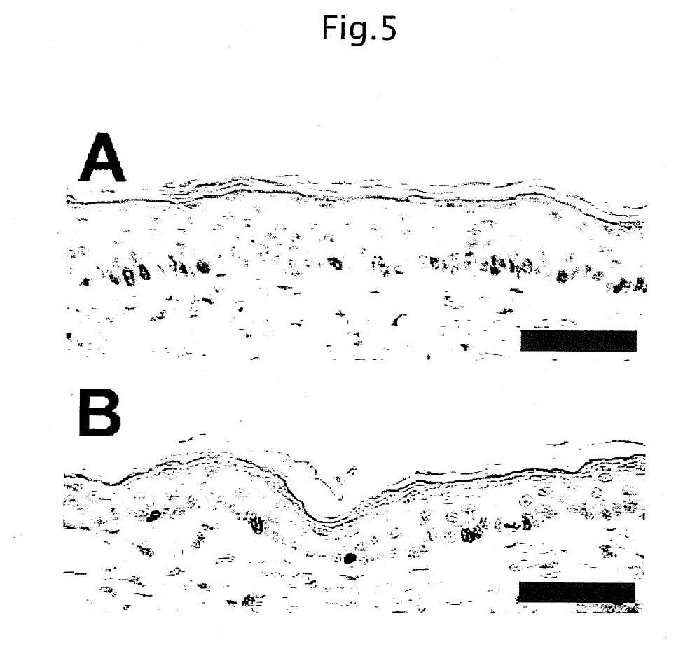

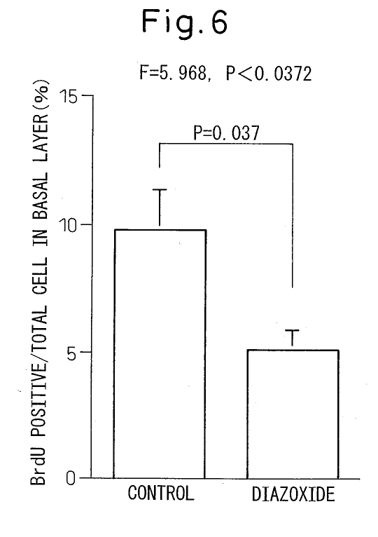

Topical application of potassium channel opener, Diazoxide prevented the epidermal hyperplasia induced by acetone treatment under low environmental (FIG. 5). The representative sections are shown in FIG. 5. FIG. 5A shows the hyperproliferative epidermis treated with water after the acetone treatment under the low humidity. Dark spots show BrdU positive cells. The increase was seen on the epidermal basal layer (FIG. 5A). Topical application of 100 uM Diazoxide prevented the epidermal hyperplasia (FIG. 5B). FIG. 6 shows quantified results shown in FIG. 5. Significant reductions of epidermal proliferation were observed on Diazoxide treated skin.

Discussion

While the mechanisms that regulate exocytosis of LB have not yet been fully clarified, LB secretion begins within 15-30 min. after barrier disruption (Elias, P. M., and Cullander C. (1998). J Invest Dermatol Symp Proc 3(2): 87-100; Menon, G. K., Feingold K. R., Elias P. M. (1992). J Invest Dermatol 98(3): 279-89). Ionic shifts in response to altered barrier status are clearly critical. For example, we previously demonstrated that influx of Ca⁺⁺ ion into keratinocytes prevented LB secretion and delayed barrier recovery, while influx of chloride ions into keratinocytes instead accelerated LB secretion and barrier recovery (Lee, S. H., et., al., op. cit.; Denda, M. et al., op. cit.). In the present study, we used two different types of potassium-channel openers and blockers, i.e., ATP- and Ca⁺⁺-dependent modulators. Each of the different types of channel openers accelerated the barrier repair, while the channel blockers delayed it. Previously, Koegel and Alzheimer, demonstrated that Ca²⁺-dependent potassium channel opener, 1-EB10, induced the membrane polarization in keratinocytes (Koegel, H. and C. Alzheimer (2001). Faseb J 15(1): 145-154.). The fact that several different drugs that decrease intracellular K⁺ accelerate barrier repair while drugs that increase intracellular K⁺ inhibit barrier repair makes it very unlikely that the observed effects are due to non-specific effects of the various compound used.

To estimate the effect of the drugs employed in this study on potassium dynamics in epidermal keratinocytes, we studied differentiated cultured keratinocytes because it is technically very difficult to measure intracellular K⁺ levels in vivo in intact skin. Cultured keratinocytes may not perfectly mimic what happens in vivo but are a model that has been widely used to study keratinocyte biology. We observed as expected that K⁺ channel openers decreased intracellular K⁺ levels. This observation would support our hypothesis that decreases in intracellular K⁺ stimulate lamellar body secretion.

In the case of neurons, which are of the same embryonic origin as keratinocytes, influx of either Ca⁺⁺ or sodium ions induces depolarization of the cell membrane, while influx of chloride ions and efflux of potassium ions induces membrane re-polarization (Shepherd, G. M., et al., op. cit.). Should the electro-physiological state of keratinocytes be similar to neurons, changes in each calcium, chloride and potassium ion could induce similar electro-physiological changes in the keratinocyte membrane. Thus, the first step in the exocytosis of LB, i.e., fusion and transition of the LB into the cell membrane, could be stimulated by electro-physiological changes of the keratinocytes cell membrane. In other cell types electro-chemical changes have been shown to alter the phase transition properties of the membrane (Ortiz, A., J. A. Killian, et al. (1999). Biophys J 77(4): 2003-14.; Binder H., Zschornig O. (2002). Chem. Phys Lipids 115(1-2): 39-61), perhaps leading to organelle fusion and exocytosis.

In summary, maintenance of a competent permeability barrier in the face of external and internal stressors requires signals between the stratum corneum (SC) interface and the metabolic machinery in the underlying nucleated epidermis. For example, reductions in Ca⁺⁺ after acute barrier disruption stimulate lamellar body (LB) secretion, a response required to restore barrier homeostasis. Though alterations in external K⁺ levels also regulate barrier recovery after acute insults, the mechanisms whereby K⁺ regulates barrier function remain unknown. Single applications of either K⁺ channel openers (i.e., 1-EB10, minoxidil, diazoxide) or the K⁺ ionophore, valinomycin, accelerate barrier recovery after acute insults to murine skin, paralleled by a reduction in intracellular K⁺ levels in CHK. In contrast, applications of K⁺ channel blockers (i.e., gilbenclamide, dequalinium) delay barrier recovery. Alterations in intracellular K⁺ regulate barrier homeostasis by either stimulating (reduced K⁺) or inhibiting (elevated K⁺) lamellar body secretion. Finally, development of epidermal hyperplasia, a down-stream consequence of barrier disruption, is also inhibited by agents that reduce intracellular K⁺ levels. These results support the idea that changes in K⁺ levels that occur after barrier disruption, signal metabolic responses; i.e., lamellar body secretion, which accelerates normalization of barrier function. More generally, these results support the concept of the keratinocyte as an electrophysiologic sensor, whereby modulations in ion levels, in response to stressors, regulate functional responses at the interface of the epidermis and the external environment.

What is claimed is:

1. A method for accelerating cutaneous barrier recovery by inducing efflux of potassium ion from an epidermal cell.

2. The method of claim 1, wherein inducement of efflux of potassium ion from an epidermal cell is attained by applying a potassium channel opener and/or a potassium ionophore on skin.

3. The method of claim 2, wherein said potassium channel opener is one or more agent selected from the group consisting of 1-EB10, minoxidil and diazoxide.

4. The method of claim 2, wherein said potassium ionophore is valinomycin.

5. A method for preventing epidermal hyperplasia induced by inducing efflux of potassium ion from an epidermal cell.

6. The method of claim 5, wherein inducement of efflux of potassium ion from an epidermal cell is attained by applying a potassium channel opener and/or a potassium ionophore on skin.

7. The method of claim 6, wherein said potassium channel opener is one or more agent selected from the group consisting of 1-EB10, minoxidil and diazoxide.

8. The method of claim 6, wherein said potassium ionophore is valinomycin.
